(19) United States
(12) Patent Application Publication
(10) Pub. No.: US 2025/0132031 A1
(43) Pub. Date: Apr. 24, 2025

(54) Implantable Medical Device and Communication Method

(71) Applicant: BIOTRONIK SE & Co. KG, Berlin (DE)

(72) Inventors: Brian M. TAFF, Portland, OR (US); Madeline Anne MIDGETT, Portland, OR (US); Brad MCMILLAN, Lake Oswego, OR (US)

(73) Assignee: BIOTRONIK SE & Co. KG, Berlin (DE)

(21) Appl. No.: 18/683,869

(22) PCT Filed: Aug. 19, 2022
(86) PCT No.: PCT/EP2022/073241

§ 371 Date: Feb. 15, 2024

Related U.S. Application Data
(60) Provisional application No. 63/242,137, filed on Sep. 9, 2021.

(30) Foreign Application Priority Data
Sep. 27, 2021 (EP) 21199027.0

(51) Int. Cl. G16H 40/67; G16H 10/60

(52) U.S. Cl. CPC G16H 40/67; G16H 10/60

(57) ABSTRACT

An implantable medical device (IMD) comprising at least one sensor, a processor and a transceiver module, wherein the at least one sensor is configured to monitor at least one predefined body function of a patient, wherein the transceiver module is configured to bi-directionally exchange messages with an external device, namely, to receive request messages from the external device and to send respective response messages to the external device replying to the request messages. In order to best leverage the limited data communication throughput of IMDs to enable body function data streaming the processor is configured to receive signals of the at least one predefined body function detected by the at least one sensor. Further, a communication system comprising the IMD, a communication method as well as a computer program product and a computer readable data carrier is also provided.

### CROSS-REFERENCE TO RELATED APPLICATIONS

This application is the United States National Phase under 35 U.S.C. § 371 of PCT International Patent Application No. PCT/EP2022/073241, filed on Aug. 19, 2022, which claims the benefit of European Patent Application No. 21199027.0, filed on Sep. 27, 2021, and U.S. Patent Provisional Application No. 63/242,137, filed on Sep. 9, 2021, the disclosures of which are hereby incorporated by reference herein in their entireties.

### TECHNICAL FIELD

The present invention is directed to an implantable medical device, in particular to a leadless medical device such as an ILP, which is part of a communication system comprising the implantable medical device (IMD) and an external device, preferably in the function of an external communication unit. The IMD is configured to monitor the health status of a patient and may further be configured to deliver a therapy signal to the patient. The IMD comprises at least one sensor configured to monitor at least one predefined body function of the patient. The body function may also be or be called physiological function. The external device is located at least partially extracorporeally. The present invention is further directed to a respective communication system, a communication method, a respective computer program product and a respective computer readable data carrier.

### BACKGROUND

Active implantable medical devices (IMDs), for example, a pacemaker (with leads), an implantable cardiac monitor (ICM), an Implantable Leadless Pacer (ILP), an Implantable Leadless Pressure Sensor (ILPS), an Implantable Cardiac Defibrillator (ICD) or a Subcutaneously Implanted Cardiac Defibrillator (S-ICD), contain sensors that collect physiological signals in order to monitor the health status of the patient and transmit the physiological signals as data to a physician device or to a remote server using the external device. The data collected from these various sensors can include, but are not limited to, ECG, intracardiac electrogram (IEGM), for example data of both atria and both ventricles, impedance, activity, posture, heart sounds, pressure, respiration, and other data. Active IMDs, e.g., pacemaker, ILP, ICD, ICM or S-ICD contains electronics and a power source, and certain of such devices may provide therapy signals to the patient, for example electrical stimulation within a heart chamber or atrium.

Usually, such IMD comprises a processor for data processing and a transceiver module configured to bi-directionally exchange messages with the external device if, for example, implanted within the body of the patient. The external device is configured for bi-directional message exchange with the transceiver module of the IMD, as well. The external device may be a separate device connected to a computer or Programmer or may be a module integrated within a remote device such as a computer or Programmer. The external device produces and sends messages to the transceiver module of the IMD in form of requests, for example for receiving data concerning the health status of the patient or IMD status from the IMD or for programming (in order to configure the IMD to apply appropriate therapies to the patient).

Processing data, applying algorithms to the data or communication with the external device on the IMD has drawbacks because it limits the extent of processing and performance of the algorithms due to the low-power requirements of small IMDs such as ILPs. High-power, high-performance communication, data processing and algorithms cannot be performed on such IMD at the risk of sacrificing device longevity. In order to extend IMD longevity to provide longer clinical benefit, larger batteries must be used, thereby increasing the physical size of the IMD, which is typically undesired. Accordingly, on the one hand, communication infrastructure and message processing need to be optimized for lowest energy consumption. On the other hand, IMD in the market provide streamed body function data, e.g., streamed IEGM data, as part of the present, standard of care. Such devices have, to date, largely represented shallow subcutaneous implants that benefit from sizeable on-board primary cells and, when paired with their shallow placements within patient anatomy benefit from wandless telemetry capabilities that support data rates sufficient to stream body function data from an organ or from a part of an organ, e.g., each heart chamber, where sensing and therapy support are rendered.

In leadless devices, the IMDs reside at greater depths within the patient anatomy and require the use of much smaller on-board power sources. Implants of this type therefore face a trade-off in either reducing the amount of data that they can relay to external devices in a given span of time or encounter needs for greater power to try and match data rates of comparable legacy devices. Body function data streaming, such as IEGM data streaming, support capabilities are therefore challenged in leadless products with regard to their service standard of care which conventional products (with leads), for example CRM products, need.

For example, a leadless IMD is known that is capable of supporting sensing of both right ventricle (RV) and right atrium (RA) signals. The known product further employs an accelerometer to detect mechanical motions of the heart associated with atrial contractions as a cue for enabling AV synchronous pacing. As a known system comprising this known leadless IMD leverages a wan-based or desired telemetry support infrastructure common to legacy cardiac rhythm management products, the IMD must exhibit substantial power output to relay signalling to a programmer as is forced to combat the attenuation effects that plague the affiliate communication scheme's high-frequency carrier across the longer in-body channel. Such a circumstance, when paired with the smaller on-board primary cell capacity of such leadless IMDs has direct consequences for enabling all the expected functionality in a follow-up procedure at the expense of real reductions in the IMD's effective longevity.

Accordingly, it is desirable to provide an IMD, a communication system and method which are improved with regard to at least the above problems, in particular which best leverage a limited data communication throughput more suitable to the power needs of deeply implanted leadless IMDs to enable body function data streaming that is clinically useful and compliant with standard of care expectations in such products.

The present disclosure is directed toward overcoming one or more of the above-mentioned problems, though not necessarily limited to embodiments that do.

### SUMMARY

At least the above problem is solved by an IMD with the features of claim 1, by a communication system with the features of claim 9, a respective communication method with the features of claim 11, by a computer program product with the features of claim 14, and by a computer readable data carrier with the features of claim 15.

In particular, at least the problem is solved by an implantable medical device (IMD) comprising at least one sensor, a processor and a transceiver module, wherein the at least one sensor is configured to monitor at least one predefined body function of a patient, wherein the transceiver module is configured to bi-directionally exchange messages with an external device, namely to receive request messages from the external device and to send respective response messages to the external device, for example in order to reply to the one request message, wherein the processor is configured to receive signals of the at least one body function detected by the at least one sensor, to process these signals thereby producing body function data and to generate each response message such that it comprises the body function data only if it was requested by a previously received request message from the external device and such that it comprises a data state field at a predefined location within the response message wherein
a) a predefined first value is assigned to the data state field if the response message does not include said body function data or
b) a predefined second value different from the first value is assigned to the data state field if the response message includes said body function data.

Power-optimal approaches may involve that an direct memory access (DMA) and ring buffer storage are turned off unless specifically requested by the Programmer to run for streaming support.

The IMD is an implantable medical device, for example a leadless IMD, as defined above configured to monitor the health status of the patient. Further, the IMD may be an IMD configured to deliver a therapy signal to the patient. For example, the IMD may be an ILP for implantation within an atrium or a ventricle of a patient's heart.

In one embodiment the body function signals are IEGM signals, for example RA signals and/or RV signals and the predefined body function data are IEGM data, for example RA data and/or RV data.

The IMD comprises a processor for data processing and a transmitter module or a transceiver module (e.g., an antenna or a magnetically-coupled inductive coil) for sending and transmitting messages (i.e., communication signals) to the external device, namely, to receive request messages from the external device and to send respective response messages to the external device. Messages are bit strings embedded in a system of syntax rules and semantics rules defined in a communication protocol which are expressed in and/or understood/interfaced with/by respective algorithms and data structures. The request messages received by the transceiver module are transmitted to the processor for data processing. Conversely, the processor of the IMD generates the content of the signals/messages which are then transmitted to the transceiver module for sending/transmitting to the external device. The IMD further comprises at least one sensor which is configured to monitor at least one predefined body function of a patient and therefore receives its respective signals, for example IEGM signals, e.g., RA signals and/or RV signals.

The IMD may comprise further modules such as a memory for storing data (e.g., a storage buffer), a power supply such as a battery, at least one signal generator for generating, for example, electrical or electromagnetically therapy signals in order to provide the therapy to the patient. The transceiver module, the memory, the power supply, the at least one sensor and/or the signal generator may be electrically connected to the processor.

The memory of the IMD may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device.

The external device is located at least partially extracorporeally and may be a separate module wirelessly or by-wire connected to a computer (e.g., physician device, remote server, Programmer) having a processor. Alternatively, the external device may be an integrated unit of such computer. The computer may be located at least partially extracorporeally. For bi-directional communication the external device may comprise a transceiver or transmission module for messages (signals), for example, an antenna or a magnetically coupled inductive coil. The communication module may comprise a processor, in particular, if it is a module separate from a computer.

In one embodiment the communication of the external device and the IMD may be wireless communication via the patient's body and/or the air using electromagnetic waves, for example Bluetooth, WLAN, ZigBee, NFC, Wibree or WiMAX in the radio frequency region, or IrDA or free-space optical communication (FSO) in the infrared or optical frequency region. Communication by wire (electrical and/or optical communication) may be possible, as well.

For deep leadless implants the preferred means for communicating by/with the IMD and the external device is by conducted, acoustic, magnetic inductive, and potentially optical methods. If the external device is a patient device it may be connected to a Programmer by means of other wireless capabilities that could include Bluetooth, etc. If the external device is a patient device it may be connected to a remote service center through WLAN, cellular networks, or the like. If the external device is simply the wand/programmer head of the Programmer, it is simply a wired connection that links it to the Programmer.

With regard to the present invention each processor is regarded as a functional unit of the IMD, the unit and the computer, respectively, that interprets and executes instructions comprising an instruction control unit and an arithmetic and logic unit. The remote computer, i.e., a functional unit that can perform substantial computations, including numerous arithmetic operations and logic operations without human intervention, such as, for example, a personal mobile device (PMD), a desktop computer, a server computer, clusters/warehouse scale computer or embedded system.

According to the present invention, the processor of the IMD is configured to receive signals of the at least one predefined body function detected by the at least one sensor, to process these signals thereby producing body function data requested by the external device and to generate a respective response message. The response message is generated such that it comprises not in any case predefined body function data but only if the predefined body function data were requested by a previously received request message from the external device. Further, each response message comprises a so-called data state field at a predefined location within the response message wherein
A) a predefined first value is assigned to the data state field (e.g., one bit data state field to which the value zero is assigned) if the response message does not comprise or contain said body function data or
b) a predefined second value different from the first value is assigned to the data state field (e.g., one bit data state field to which the value one is assigned) if the response message comprises or contains said body function data.

The above IMD may be used within a communication system for a wireless message transfer between the external device and the IMD. The external device may be adapted to generate and send a request message comprising a data request field to the IMD, wherein a specific value (e.g., the value one) is assigned to the data request field from which the processor of the IMD derives that the IMD is requested to provide body function data to the external device. If the data request field of the request message contains another value than the specific value (e.g., the value zero) then the processor of the IMD derives that the external device does not request sending any body function data. In one embodiment the length of the request field is 1 bit to 4 bits.

The above IMD and the communication system provides a communication packet design structure that includes appropriate flags (data state field) to report the information carried in IMD responses along with a capacity to tack blocks of body function data, such as IEGM data, onto the back end (i.e., as a caboose) of any such responses. Thereby, the data structure of the response message is adapted to the actual need of the user based on the content of the request message sent with the external device. Accordingly, only requested data are transmitted (and data not requested are avoided) which has a positive effect on the IMD's effective longevity and/or provides more spans within the communication link where other CMD/response packets can be relayed. A plurality of response messages may be sent by the transceiver module one after another thereby forming a stream of messages. Further, the external device when receiving the response message gets information about the structure of the response message (for example, the data state field which contains the information whether the message contains body function data or not, other information on the structure may be contained as well) so that a correct and reliable processing of data received with the response message within the external device or the computer connected to the unit is guaranteed. The information about the structure of the response message is further used to correctly display the body function data on a display provided with the external device or the computer.

With the expression “assigned to” it is meant that the response message transmits in its data state field at least the first value or the second value, respectively, as defined above. The same applies analogously to the request message and its data request field. Transmission of additional values within these fields is thereby not excluded but depends on the length of the respective field. In one embodiment the length of the data state field is 1 bit to 1 Byte. In case, the data state field is 1 bit, the first value may be the value 1 and the second value may be the value 0, for example. By using the data state field the IMD indicates whether the requested body function data is tacked at the response message or not.

In one embodiment the request message is a proximity request message, i.e., a regular, in predefined time intervals provided request to maintain the data link between the IMD and the external device, which is also called proximity command relay, a monitoring request message, i.e., a request for determining a certain actual body parameter, a programming message, i.e., a request for setting a certain program parameter or change a certain program parameter of the computer program for execution in the processor, a status request message, i.e., a request for providing a certain status parameter of the processor or any other module of the IMD (e.g., battery status), an execution request message, i.e., a request for execution of a certain program sequence, or a data request/interrogation message, i.e., a request for delivery of certain data stored in the data memory of the IMD.

In one embodiment the response message (message sent by the IMD to the external device) is a proximity response message, i.e., a response to a proximity request message, a reporting response message, i.e., a response to monitoring request message, a programming response message, an execution response message, or a data request/interrogation response message—all of them after finishing the sequence necessary to provide the relevant data, or an acknowledgement response message, i.e., an instantaneous response to monitoring request message, to a program request message, to an execution request message, or to a data request/interrogation message—with regard to all of them only telling that the IMD received the respective request.

Each of the above mentioned messages (request messages, response messages) may be a full message containing all necessary components and fields according to the respective communication protocol or may form only a part of a full message because the predefined maximum transmission unit was reached. In the latter case the full message is divided into several packets which are separately sent. One field (e.g., the data state field or the data request field) is a section of the message bit string having a predefined length (e.g., 1 to 4 bits or 1 bit to 1 byte) and/or a predefined location within the full bit string. The message may naturally include other details such as sync bytes, addressing information, command and response content but such content have been deliberately excluded from the present discussion as it is part of a broader protocol but not specifically relevant to the focus of the present disclosure.

In one embodiment the data state field comprises further information on at least one property of the comprised body function data such as, for example, a type of said body function data and/or a length of the body function data and/or its data resolution and/or an information regarding a body function data structure within the response message (While the data state field may contain such information, the plan for relaying such content may be part of the marker types installed within the IEGM data). In this embodiment, the property of the body function data described in the data state field tells the external device on the one hand information about the structure of the response message. For example, from the information about the length of the body function data (i.e., the number of bits) the external device may determine the end of the message block (field) belonging to the body function data. In one embodiment a cyclic redundancy check (CRC) word may be included that accounts for this length information such that the external device side infrastructure could be sure to properly time and stitch together any received data for display to the user. On the other hand, the information about the at least one property of the comprised body function data allows the external device or a computer connected to the external device to correctly assess and display the received data of the at least one body function. The type of the predefined body function data refers to the kind of transmitted data if the IMD monitors different body functions, for example, RA data and RV data. As indicated above, the information on at least one property of the comprised body function data comprises the resolution of the data (e.g., 128 Hz, 64 Hz). The system, if not user, may access controls to support rendering either a selected single IEGM data at high resolution (e.g., RA or RV data) or the simultaneous display of two IEGM data at low resolution (e.g., RA and RV data). This provides system controls (which may be user accessible depending on the product design) to configure the resolution of streamed content of more than a single body function source in the IMD, e.g., of more than a single IEGM source in leadless IMD. This may effectively render standard of care CRM capabilities despite the lower-data rate demands necessary for low-power transmission from/to deep IMD like ILP. Further, if the display of a specific source is specifically desired (e.g., as part of an atrial far-field sensing test), the inventive approach offers a means for optimizing/boosting its resolution. Alternatively, in cases where a balanced approach is sought, the approach supports tuning streamed resolutions to report clinically relevant signaling without overtly compromising the interpretability of any signal source. Accordingly, this embodiment offers the ability of the system to alter the presented body function stream resolutions and format subject to follow-up use case needs (e.g., high-resolution atrial streaming during an atrial sensing test, and low-resolution rendering otherwise).

In one embodiment the data state field further comprises information on a time point at which the comprised body function data changes at least one of its at least one property. In this embodiment it is, for example, allowed rendering two different resolutions or different data types of the body function adapted to the user's needs within one message. It is, for example, possible to change the transmission of a selected single IEGM data at high resolution (e.g., RA or RV data) to the simultaneous display of two IEGM data at low resolution (e.g., RA and RV data) and vice versa.

Additionally or alternatively, a marker is installed in the IEGM at the sample location where the data changes (instead in the data state field). The position of the sample then effectively communicates the time.

In one embodiment the response message, for example the body function data, is used to carry any event markers associated with the body function data (which can actually be used to tell when the body function data changes in source), for example with the IEGM data. They are transmitted, for example, within the IEGM payload in an embedded manner where one or more bytes may be used to report, for example, an atrial sense event (e.g., an atrial systole), a ventricular sense element (e.g., a ventricular systole), a ventricular extra-systole, or otherwise.

In one embodiment the processor is electrically connected to a storage buffer, wherein the processor is adapted such that body function data are directly and continually stored in the storage buffer after receiving a request to provide at least one body function data from the external device. To save on overhead associated with the management of body function stream data (e.g., IEGM data), an embodiment for supporting the needs outlined above leverages a hardware-based accelerator to enable direct memory access (DMA). In other words, an “engine” within the integrated circuitry of the processor facilitates the rapid packing of sensed data into a storage buffer. In one embodiment such a storage location is a rotating ring buffer of sufficient size that is continually filled when body function data is requested by the external device. Subject to incoming requests from the external device side infrastructure to either respond to a command or maintain the communication (proximity response message) the IMD then “rips” all the new content stored within the ring buffer since its last “dump” operation (i.e., since the preceding command/response interaction). Generally, the processor may be configured such that the complete content of body function data of the storage buffer, which has been stored in the storage buffer since last emptying operation, is got from the storage buffer and transferred to the processor.

Analogously, at least the above problem is solved by a communication method of an IMD comprising at least one sensor, a processor and a transceiver module, wherein the at least one sensor monitors at least one predefined body function of a patient, wherein the transceiver bi-directionally exchanges messages with an external device, comprising the following steps:
receiving a request message from the external device,
checking whether the received request message comprises a specific value assigned to the data request field from which the IMD derives that the IMD is requested to provide body function data to the external device,
receiving signals of the at least one predefined body function detected by the at least one sensor, for example IEGM signals, e.g., RA signals and/or RV signals,
processing these signals thereby producing body function data requested by the external device, for example IEGM data, e.g., RA data and/or RV data,
generating a response message such that it comprises the body function data only if it was requested by the previously received request message (i.e., only if the previously received request message comprised the specific value in the data request field) and such that it comprises a data state field at a predefined location within the response message wherein
a) a predefined first value is assigned to the data state field if the response message does not comprise or contain said body function data or
b) a predefined second value different from the first value is assigned to the data state field if the response message comprises or contains said requested body function data,
transmitting the generated response message to the external device by the transceiver module thereby replying to the request message.

The above method has the same advantages as the above IMD or communication system. Further, the transmitted body function data are used for further processing and/or displaying in/at the external device or in the connected computer. The further processing may include adaption of program parameters, data transfer to a health care practitioner for data assessment in order to adapt the respective patient's therapy.

In one embodiment body function data are directly and continually stored in a storage buffer of the IMD after receiving a request containing the specific value in the data request field from the external device.

In one embodiment of the above method the complete content of body function data of the storage buffer, which has been stored in the storage buffer since last emptying operation, is transferred to the processor.

Additionally or alternatively, the processor is there to assemble the packet and ship it out as a response. It may simply point to or grab the IEGM from the ring buffer and shove it into the outbound message.

The above method is, for example, realized as a computer program which comprises instructions which, when executed, cause the processor to perform the steps of the above method (to be executed by the IMD, in particular at its processor and transceiver module) which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by a processor, cause the processor to perform the steps of the above defined method. Accordingly, a computer readable data carrier storing such computer program product is disclosed.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein:

FIG. 1 shows an embodiment of the inventive communication system comprising an implantable leadless pacemaker (ILP) and an external device, wherein the ILP is shown within a cross section of a patient's heart,

FIG. 2 depicts a scheme showing body function signals in the form of RV IEGM signals and respective response messages of a first embodiment of an inventive communication method,

FIG. 3 depicts a scheme showing body function signals in the form of RA and RV IEGM signals and respective response messages of a second embodiment of an inventive communication method,

FIG. 4 depicts a scheme showing body function signals in the form of RA and RV IEGM signals and respective response messages of a third embodiment of an inventive communication method, and

FIG. 5 depicts a scheme showing body function signals in the form of RA and RV IEGM signals and respective response messages of a fourth embodiment of an inventive communication method.

### DETAILED DESCRIPTION

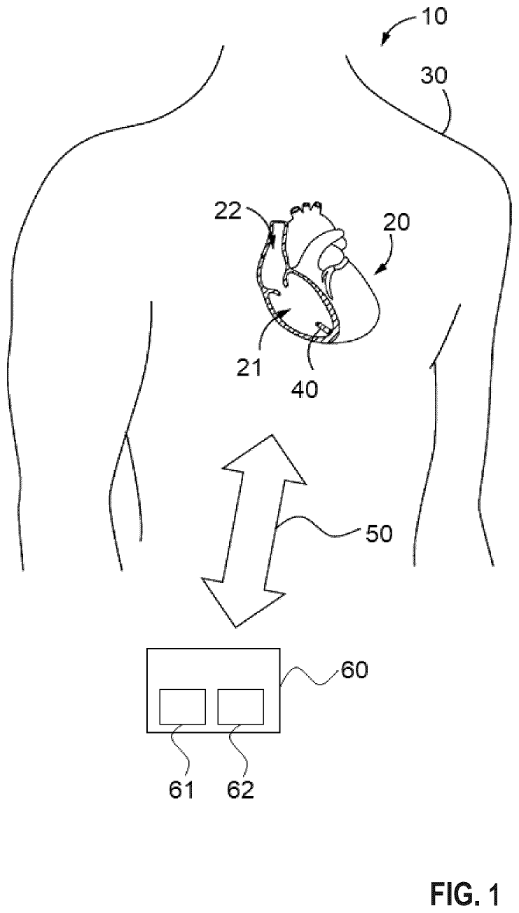

FIG. 1 shows an example communication system 10 and a heart 20 (with right ventricle 21 and right atrium 22) of a patient 30. The system 10 comprises a leadless ventricular pacemaker device 40 (hereinafter “ILP 40”) as an example for an IMD and an external device 60. The ILP 40 may be configured to be implanted within the right ventricle 21 of the heart and to pace this ventricle as well as sense intrinsic ventricular depolarization. The ILP 40 may further comprise an accelerometer sensor in order to measure mechanical motions of the heart 20 associated with atrial contractions as a cue for enabling AV synchronous pacing. A programmer (not shown) may be used to program ILP 40 using the external device 60. The external device 60 is located outside the body and is adapted to communicate bi-directionally with the ILP 40.

The ILP 40 may comprise modules such as a processor, a data memory, a signal generator unit for providing treatment signals (e.g., pacing signals), a sensor comprising an IEGM measuring unit for sensing ventricular depolarization and the accelerometer sensor, a transceiver module for sending and receiving messages to the external device 60, and a power source wherein the modules are electrically connected to each other. The power source may include a battery, e.g., a rechargeable or non-rechargeable battery. The data memory may include any memory type mentioned above.

The external device 60 comprises a processor 61 and a transceiver 62 for exchange of messages with the ILP 40 which are electrically connected with each other. Further, the external device 60 may exchange messages with a Programmer and/or a remote computer (not shown) and may be integrated within the Programmer or remote computer. In the latter case the processor 61 may be integrated within the processor of the Programmer or the computer. The bi-directional exchange of messages with the ILP 40 is symbolized by the double arrow 50. The leadless communication of the external device 60 with the ILP 40 may be facilitated by acoustic, conducted, or magnetic induction methods or electromagnetic waves in the radio-frequency range, for example.

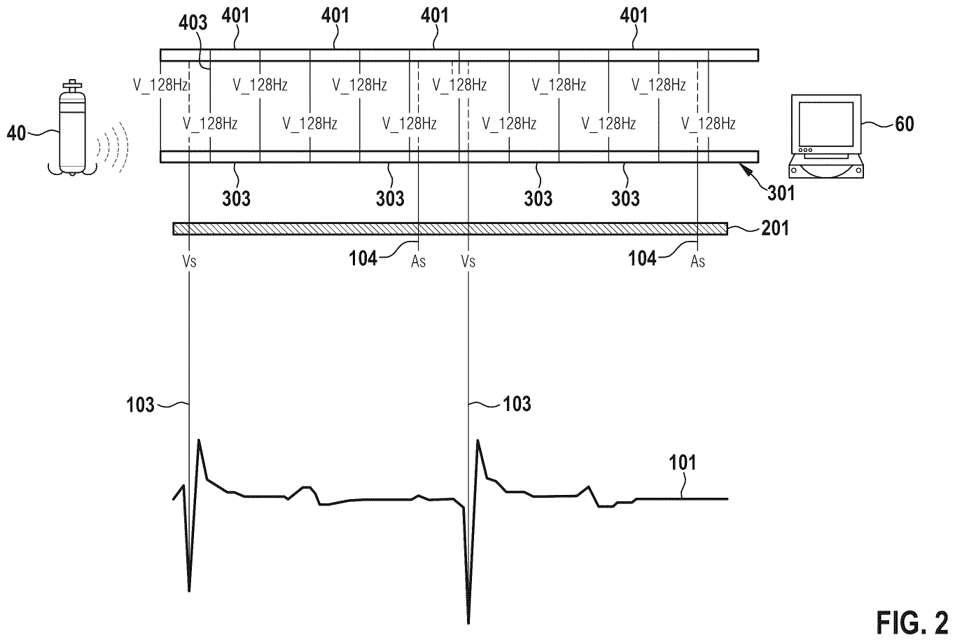

In the following the operation of one embodiment of the communication method is explained using FIGS. 2 to 5. The series of FIGS. 2 to 5 offers added scrutiny of the interactions used to support the general, disclosed approach to managing and reporting high- and low-resolution IEGM streams. Within FIGS. 2 to 5 similar reference numbers refer to similar elements of the schemes.

FIG. 2 shows high resolution streaming of an IEGM signal, in particular a RV signal 101 by the respective sensor of the ILP 40 comprising events like ventricular systoles 103 and atrial systoles 104. Atrial systoles are very often not visible within the RV stream. The marker “As” was generated behind the scenes using the atrial auto-detection circuitry which leverages signaling distinct from the RV stream. The RV signal 101 is continuously measured or collected by the ILP sensor and transmitted to the processor. Thereby, an analog signal is taken and converted into a digital value which then gets placed within the IEGM stream. The processor generates a high-resolution (e.g., 128 Hz) RV data stream 301 solely from a single data type 201 that is packed into the data memory formed by a DMA ring buffer formed by a plurality of packets 303 (comprising body function data). Packets (i.e., 303) are or may be simply the content pulled from the ring buffer between its start and end point indices at any point in time. Each time content is pulled from the buffer to ship to the external device, it makes the IEGM payload for the packet. For transmission to the external device 60 the data packets 303 are ripped from the DMA ring buffer forming a plurality of response messages 401 each containing a predefined second value in a data state field indicating that each response message 401 comprises RV data as body function data. In other words, the contents of the ring buffer are only ever placed into a packet if the response packet is one where the programmer has specifically requested the IEGM be relayed as part of the response. The response packet would include a data state field that indicated it contained IEGM. Further, the respective messages 401 comprise the event data (time point of the ventricular systole 103 and of the atrial systole 104) embedded within the RV data. Thereby, the sample's position within the packet is or may be what determines the time point of the event, and the type of the marker indicates what event occurred. Further, any message 401 sent from the ILP include a packet start marker 403 that not only reports the type of the sent IEGM (e.g., RV type data), but also the resolution at which it is recorded (e.g., 128 Hz). This approach may be adopted to only render high-resolution content from a number of sources (e.g., RV stream 101).

After transmission of messages 401 to the external device 60 the RV data stream derived from the messages 401 may be displayed at a display of a Programmer or of a computer or may be further processed. The external device 60 may be integrated within the Programmer or the computer.

Within FIGS. 2 to 5, all messages 401 are presented as being the same length. Such a condition is not, in fact, the case in a routine programmer communication session where command requests are sent in nominally asynchronous fashion. The shown conditions are more representative of conditions where the communication link is simply maintained by means of proximity commands which are sent, for example, every 200 ms as a means to keep the communication channel between the ILP 40 and the Programmer (and the external device 60) in an active state.

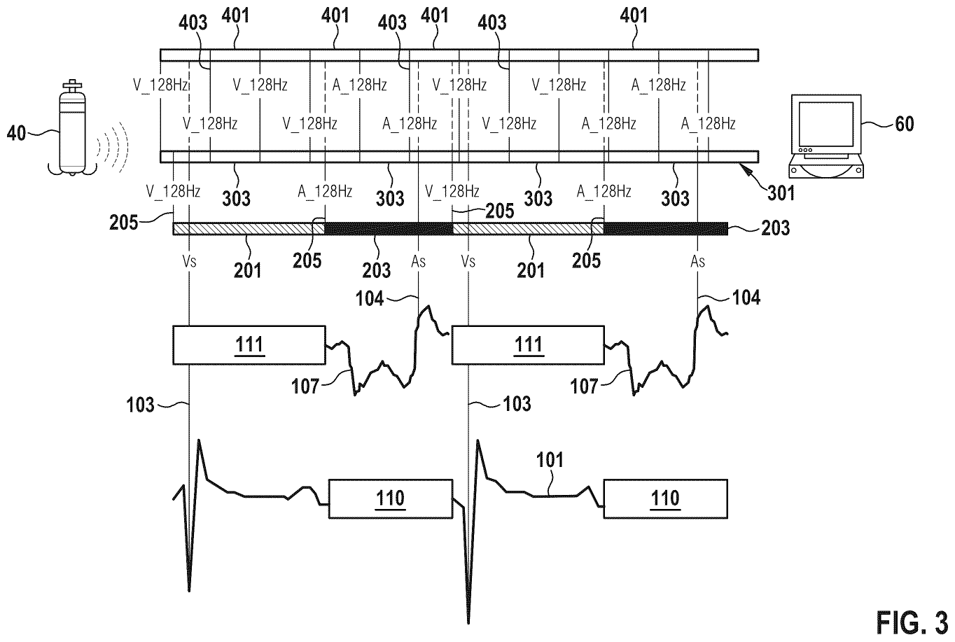

To support conditions where one provides targeted transitions between specific distinct IEGM sources (e.g., between atrial only or ventricular only) the outlay in FIG. 3 proves descriptive. The scheme illustrates the RV stream 101 measured as indicated above and the RA stream 107 detected continuously by the accelerator. In the sections 110 of the RV stream 101 and in the sections 111 of the RA stream as viewable by the user the external device may use the content from the rendered channel at this time (measurement values of RV stream 101 in section 111 or measurement values of RA stream 107 in section 110 of the RV stream 101) and display it on the other (e.g., RV data 101 rendered into RA channel or RA data 107 rendered into RV channel) by either scaling it down (e.g., RV data scaled down to RA amplitudes) or up (e.g., RA data scaled up to RV amplitudes). This signal display management is handled exclusively by the external device (i.e., Programmer GUI display) and the implant plays no role in making content in 111 or 110 visible to the user. Accordingly, a continuous data stream 301 (comprising body function data) of single packets 303 is provided comprising RV type data 201 and alternating RA type data 203. The mentioned data stream 301 and the packets 303 are body function data. Such approach would avoid presenting any false information, but admittedly not be guaranteed to show all crucial elements in the raw signaling that might be tied to the reported cardiac event marker stream. As indicated by the packet start markers 403 of the messages 401 the RV data and RA data are provided in high resolution (e.g., 128 Hz). The information regarding the change of data type at as indicated in sample 205 is embedded within the RV and RA data. Similar to the embodiment shown in FIG. 2, any message 401 sent from the ILP include a packet start marker 403 that not only reports the type of the sent IEGM (e.g., RA type data or RV type data), but also the resolution at which it is recorded (e.g., 128 Hz).

FIG. 3 represents a case where the maximum resolution is shown for each channel. This may offer benefit for specific follow-up cases, but is not necessarily guaranteed to satisfy all users or the market for all conditions. As with the data described with regard to FIG. 2, the RV and RA data may be displayed after transmission to the external device 60. In addition, it should be mentioned that at the sections 110, 111, within the graphic, creative measures might be adopted by the Programmer GUI design to render display elements to the user/clinician that cover the data gap (in other words, the streamed content from the implant provides no data in regions 110 and 111). Incarnations of this type could include approaches as wide-varying as simply leaving gaps in the display (i.e., showing nothing since, as mentioned, no data fills those spaces), accentuating the display in ways that show a feature indicative of a “no data” condition (e.g., inserting a “grayed out” condition or a series of ellipses), or the scaling of alternate channel data as described in the preceding paragraph.

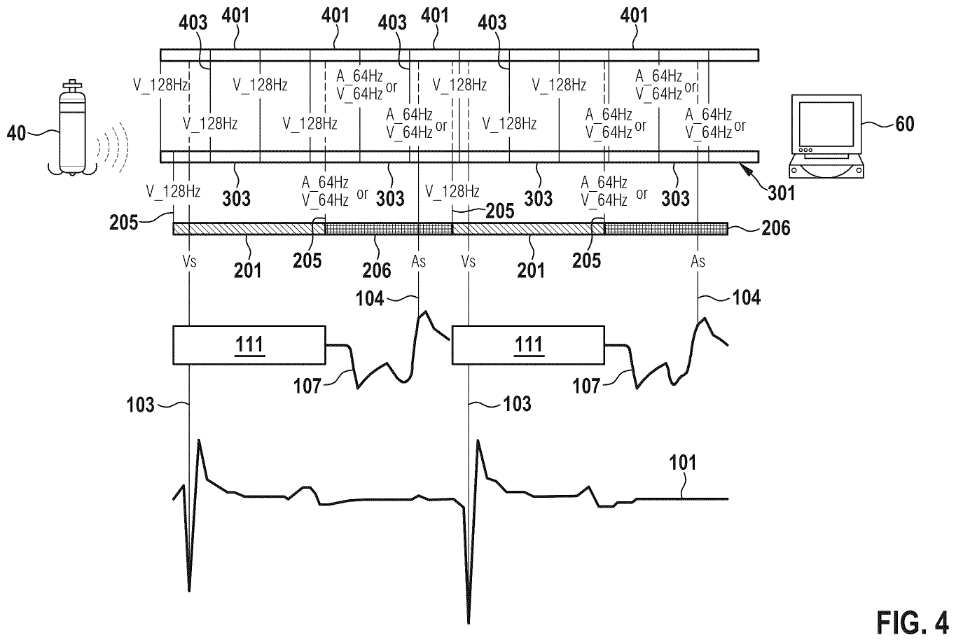

FIG. 4 depicts a condition where the system swaps between the presentation of high-resolution IEGM content on a single channel (i.e., RV stream data 101 subject to sections 201) and low-resolution IEGM content (of e.g., 64 Hz) on multiple channels (i.e., RV stream data 101 and RA stream data 107 subject to sections 206). The information on data type change and resolution change as indicated in sample 205 is embedded in the RV and RA data of the respective messages 401. Further, if applicable, at the beginning of each data packet not only the two different data types but also the resolution for each data type is given with the respective data start marker 403.

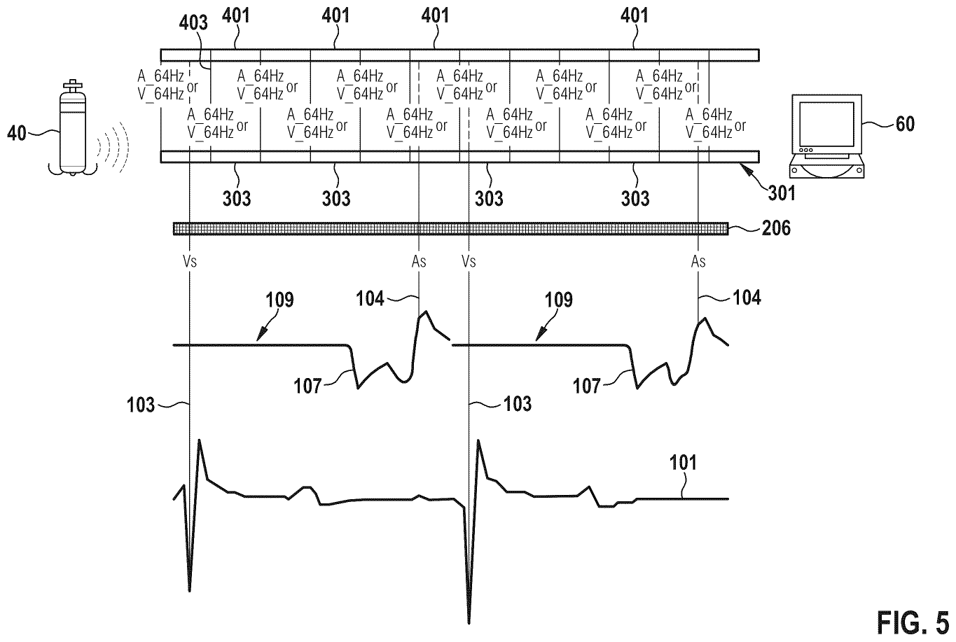

The final scheme shown in FIG. 5 even supports the simultaneous streaming of low-resolution IEGM data from multiple channels (RV data 101 and RA data 107) at all times (see data type representation 206). Sampling of multiple channels even at 64 Hz is clinically still useful for atrial and ventricular cardiac signals.

Aside from the embedded markers with the packages, each data sample within the stream may oscillate between an RA and an RV sample. In other words, sample 5 is an RA data point, sample 6 is an RV data point, sample 7 is an RV data point, etc.

All messages 401 depicted in FIGS. 2 to 5 contain a one bit data state field to which the second value is assigned (e.g., value 1) thereby indicating for the external device 60 that the respective messages 401 contains body function data (RA data 107 and/or RV data 101). Further, each message 401 may contain an information regarding the length of the contained RA data 107 and/or RV data 101.

Prior to transmission of the above explained response messages 401, the external device 60 has sent a request message containing a data request field with the length of 1 bit. The value 1 was assigned to the data request field thereby indicating to the ILP 40 that data transmission of RA and/or RV data is requested. In one embodiment, the data request field may indicate the type of data and its resolution. Triggered by this request, the ILP 40 starts measuring and/or collecting the requested data, processing and transmitting the data as described above. As soon as sufficient data are received or the clinician turns the IEGM-collection or measurement OFF, the external device 60 may send another request message in which the data request field is assigned with the value 0 thereby indicating that data generation and transmission shall be finished.

In summary, the inventive system and method provides system controls (which may be user accessible depending on the product design) to configure the resolution and type of streamed content of more than one single IEGM source in leadless products such as ILP.

Despite the above remarks on clinically meaningful signaling output, it should be noted that given the lack of real data outside of the active atrial detection windows accessible in the atrial channel during VDD mode operation there is substantial likelihood that significant portions of the collected data will represent “flat line” zero amplitude conditions. Reporting such values, while reflective of the observable conditions on the atrial channel, do not necessarily represent the best use of accessible cross-product signal resolution for the data transmission throughput supported by the leadless product. As such, many might favor a FIG. 4 configuration over a FIG. 5 rendition, as any conditions where atrial sense windows in the VDD time, can avoid occupying data space in relayed IEGM signaling in favor of higher resolution ventricular sensing.

A possible extended implementation of the concepts above (not shown) could be to pause DMA transfers to the ring buffer in segments of the stream where no meaningful data could be collected (e.g., in a possible FIG. 2b consideration during the quiet “zeros” portions) and use appropriate markers to manage intended coordination with the external device. This approach could save on data throughput limitations (i.e., by avoiding sending long streams of “zeros”) between the implant and the external device yet the time resolution of any event markers within such “non-strobed” periods would be lost (i.e., the position of “As”, “Vs”, etc. events within the non-signal portions would not be known with the precision as in prior embodiments because their position within the relayed samples would not be indexed by surrounding null data points.

Again, how the changes in observable resolution enabled by FIG. 4 are reported to the users is the subject of GUI design of the Programmer or computer which receive the data from the external device. Even the extended embodiment may lean on Programmer “smarts” to take the lower resolution markers and attempt alignment with morphological considerations within the ventricular signal.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable medical device (IMD) comprising: at least one sensor, a processor and a transceiver module, wherein the at least one sensor is configured to monitor at least one predefined body function of a patient, wherein the transceiver module is configured to bi-directionally exchange messages with an external device, namely to receive request messages from the external device and to send respective response messages to the external device, wherein the processor is configured to receive signals of the at least one predefined body function detected by the at least one sensor, to process these signals thereby producing body function data and to generate each response message such that it comprises the body function data only if it was requested by a previously received request message from the external device and such that it comprises a data state field at a predefined location within the response message wherein:
a) a predefined first value is assigned to the data state field if the response message does not comprise said body function data, or
b) a predefined second value different from the first value is assigned to the data state field if the response message comprises said body function data.

2. The IMD of claim 1, wherein the length of the data state field is 1 bit to 1 Byte.

3. The IMD of claim 1, wherein the generated response message is a proximity response message or a direct response message to a request message.

4. The IMD of claim 1, wherein the body function signals are IEGM signals, for example RA signals and/or RV signals and the body function data are IEGM data, for example RA data and/or RV data.

5. The IMD of claim 1, wherein the response message comprises event information and/or the data state field comprises further information on at least one property of the comprised body function data such as, for example, a type of said body function data and/or a length of the body function data and/or its data resolution and/or an information regarding a body function data structure within the response message.

6. The IMD of claim 5, wherein the data state field further comprises information on a time point at which the comprised body function data changes at least one of its at least one property.

7. The IMD of claim 1, wherein the processor is connected to a storage buffer, wherein the processor is configured such that body function data are directly and continually stored in the storage buffer after receiving a respective request from the external device.

8. The IMD of claim 1, wherein the processor is adapted such that the complete content of body function data of the storage buffer, which has been stored in the storage buffer since last emptying operation, is collected from the storage buffer and transferred to the processor.

9. A communication system for a wireless message transfer between an external device and the IMD of claim 1.

10. The communication system of claim 9, wherein the external device is adapted to generate and send a request message comprising a data request field to the IMD, wherein a specific value is assigned to the data request field from which the processor of the IMD derives that the IMD is requested to provide body function data to the external device.

11. A communication method of an implantable medical device comprising at least one sensor, a processor and a transceiver module, wherein the at least one sensor monitors at least one predefined body function of a patient, wherein the transceiver bi-directionally exchanges messages with an external device, comprising the following steps:
receiving a request message from the external device,
checking whether the received request message comprises a specific value assigned to the data request field from which the IMD derives that the IMD is requested to provide data of the at least one body function to the external device,
receiving signals of the at least one body function detected by the at least one sensor, for example IEGM signals, e.g. RA signals and/or RV signals,
processing these signals thereby producing requested body function data, for example IEGM data, e.g. RA data and/or RV data,
generating a response message such that it comprises the body function data only if it was requested by the previously received request message and such that it comprises a data state field at a predefined location within the response message wherein
a) a predefined first value is assigned to the data state field if the response message does not comprise said body function data or
b) a predefined second value different from the first value is assigned to the data state field if the response message comprises said body function data,
transmitting the generated response message to the external device thereby replying to the request message.

12. The communication method of claim 11, wherein body function data are directly and continually stored in a storage buffer of the IMD after receiving a request containing the specific value in the data request field from the external device.

13. The communication method of claim 11, wherein the complete content of body function data of the storage buffer, which has been stored in the storage buffer since last emptying operation, is transferred to the processor.

14. A computer program product comprising instructions which, when executed by a processor, cause the processor to perform the steps of the method according to claim 11.

15. Computer readable data carrier storing a computer program product according to claim 14.
